(19) United States
(12) Patent Application Publication
(10) Pub. No.: US 2026/0097979 A1
(43) Pub. Date: Apr. 9, 2026

(54) SILICA FERTILIZATION SYSTEMS AND METHODS

(71) Applicant: Lillianah Technologies Inc., Spring, TX (US)

(72) Inventor: Benjamin Sidney Slotnick, Spring, TX (US)

(21) Appl. No.: 19/349,387

(22) Filed: Oct. 3, 2025

Related U.S. Application Data
(60) Provisional application No. 63/703,044, filed on Oct. 3, 2024.

(51) Int. Cl. C02F 1/68; C02F 1/00; C02F 3/00; C02F 3/34; C02F 103/00; C12M 1/00

(52) U.S. Cl. CPC C02F 1/686; C02F 1/008; C02F 1/687; C02F 3/006; C02F 3/348; C12M 43/00; C02F 2103/007; C02F 2201/008; C02F 2305/06

(57) ABSTRACT

An environmental treatment distribution system includes one or more silica containers, one or more sensors configured to generate data indicative of one or more characteristics of water at a target location within an aquatic environment, and a controller. The controller is configured to determine one or more desired parameters for a silica solution based on the one or more characteristics of the water at the target location; apply a fluid to a silica source within a silica container of the one or more silica containers to wash the silica source, dilute the silica source, or any combination thereof to form the silica solution having the one or more desired parameters; and generate instructions to distribute the silica solution having the one or more desired parameters into the water at or near the target location.

### CROSS-REFERENCE TO RELATED APPLICATION

This application claims priority to and the benefit of U.S. Provisional Application No. 63/708,044, entitled “Silica Fertilization Systems and Methods,” filed Oct. 3, 2024, which is incorporated by reference herein in its entirety for all purposes.

### FIELD OF DISCLOSURE

The present disclosure relates generally to environmental treatment techniques. More specifically, embodiments of the present disclosure relate to systems and methods to distribute silica into volumes of water, such as aquatic and/or marine environments.

### BACKGROUND

The oceans are vast bodies of water covering approximately 70 percent of the surface of Earth, and approximately 99 percent of a volume of space on Earth that allows for life is in the oceans. A wide variety of marine life including plants and animals live throughout the oceans. Many parts of the oceans have the potential to harbor more, or different, types of life than they currently do. In these areas, quantity of living organisms, total biomass, and/or biodiversity is limited by low concentrations or a complete absence of specific nutrients and/or materials. Many approaches have been proposed and/or attempted to alleviate these conditions, either temporarily or permanently, but these approaches are not entirely effective or are prohibitively expensive.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of possible forms of the subject matter. Indeed, the subject matter may encompass a variety of forms that may be similar to or different from the embodiments set forth below.

In certain embodiments, an environmental treatment distribution system includes one or more silica containers, one or more sensors configured to generate data indicative of one or more characteristics of water at a target location within an aquatic environment, and a controller. The controller is configured to determine one or more desired parameters for a silica solution based on the one or more characteristics of the water at the target location; apply a fluid to a silica source within a silica container of the one or more silica containers to wash the silica source, dilute the silica source, or any combination thereof to form the silica solution having the one or more desired parameters; and generate instructions to distribute the silica solution having the one or more desired parameters into the water at or near the target location

In certain embodiments, a method of operating an environmental treatment distribution system includes receiving, at one or more processors, one or more characteristics of water at a target location within an aquatic environment. The method also includes receiving, at the one or more processors, one or more respective parameters of multiple silica sources in multiple silica containers supported by an aquatic vessel. The method further includes determining, using the one or more processors, one or more desired parameters for a silica solution based on the one or more characteristics of the water at the target location. The method further includes selecting, using the one or more processors and based on the one or more characteristics of the water at the target location and the one or more respective parameters of the multiple silica sources in the multiple silica containers, a first silica source of the multiple silica sources. The method further includes instructing, using the one or more processors, a processing system to process the first silica source of the multiple silica sources to adjust the one or more respective parameters of the first silica source of the multiple silica sources to form the silica solution having the one or more desired parameters. The method further includes instructing, using the one or more processors, distribution of the silica solution into the water at or near the target location.

In certain embodiments, an environmental treatment distribution system includes a silica container supported on an aquatic vessel within an aquatic environment and a controller. The controller is configured to generate instructions to, as the aquatic vessel travels within the aquatic environment: receive one or more characteristics of a target location within the aquatic environment; determine an arrival time of the aquatic vessel to the target location; predict one or more desired parameters for a silica solution based on the one or more characteristics of the target location and the arrival time; and control a processing system to apply a fluid to a silica source in the silica container to form the silica solution with the one or more desired parameters. The controller is also configured to generate instructions to, in response to the aquatic vessel reaching the target location at or near the arrival time, control one or more output devices to distribute the silica solution into the water at the target location.

Various refinements of the features noted above may be undertaken in relation to various aspects of the present disclosure. Further features may also be incorporated in these various aspects as well. These refinements and additional features may exist individually or in any combination.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a schematic illustration of an environmental treatment distribution system that includes a silica system, in accordance with embodiments the present disclosure;

FIG. 2 is a schematic illustration of components of the environmental treatment distribution system, in accordance with embodiments the present disclosure;

FIG. 3 is a schematic illustration of the environmental treatment distribution system implemented on an aquatic vessel, in accordance with embodiments the present disclosure; and FIG. 4 shows an example of a dispersal pattern of materials distributed from the environmental treatment distribution system of the aquatic vessel, in accordance with embodiments the present disclosure.

### DETAILED DESCRIPTION

One or more specific embodiments of the present disclosure will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure. Further, to the extent that certain terms such as parallel, perpendicular, and so forth are used herein, it should be understood that these terms allow for certain deviations from a strict mathematical definition, for example to allow for deviations associated with manufacturing imperfections and associated tolerances.

When introducing elements of various embodiments of the present disclosure, the articles “a,” “an,” and “the” are intended to mean that there are one or more of the elements. The terms “comprising,” “including,” “having,” and “based on” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

It is estimated that more than half of the oxygen in the atmosphere originates from phytoplankton, such as silica-based diatoms, that reside in the oceans. It is also estimated that at least 95 percent (and possibly 97 to 99 percent) of all carbon held in short-term reservoirs resides within the ocean. Further, it is presently recognized that nutrient cycling, such as with silica and/or other nutrients, may enable the oceans to support more and/or different types of life, as well as related biogenic sedimentation. For example, the biogenic sedimentation may include remains of algal material, such as organic-rich sediments that include carbon.

Provided herein are environmental treatment distribution techniques that promote the intentional change of conditions of aquatic environments (e.g., coastal marine, nearshore marine, open ocean, deep ocean and/or lakes, ponds, rivers, streams, bays, wetlands and estuaries, marine environments), via controlled distribution of silica (e.g., as silica dioxide, SiO₂). As described herein, the silica may be distributed with other nutrients (e.g., one or more other nutrients; sodium oxide, Na₂O), such as to constrain pH (e.g., resist acidification of water) and provide other parameters to support life. For example, a Bjerrum plot of a silica-based acid-base system in the ocean demonstrates that there is a pH variable for silicic acid (H₄SiO₄) and a silicate anion (H₃SiO₄—). Further, the silica may be distributed with other nutrients (e.g., iron, such as from iron cuttings, such as directly from iron ore, such as to mimic an iron source that has no human alteration), such as to strengthen activity of aquatic organisms (e.g., phytoplankton) in the aquatic environments. Advantageously, this is in line with and/or may mimic natural processes, such as volcanic ash and/or dust (e.g., from the Saharan Desert and/or China) inducing healthy algal blooms in our oceans today. Further, dispersion and/or fertilization techniques with silica may facilitate the increase and/or decrease of relative and/or absolute abundance of various aquatic organisms to achieve desired changes in relative and/or absolute abundance of various aquatic organisms, and/or to achieve other desired outcomes. The dispersion and/or fertilization techniques with silica may be entirely or partially intended to induce an effect, condition, change, lack of change, or stasis in an aquatic environment or in an environment, ecology, place, or system impacted by an aquatic environment. The effect, condition, change, lack of change, or stasis may be local, regional, or systemic.

In certain embodiments, the environmental treatment distribution techniques operate to decrease aquatic hypoxia, increase aquatic oxygenation, increase export of materials to depth, and/or enhance fisheries. In certain embodiments, the environmental treatment distribution techniques operate to treat harmful algal bloom, red tide, nutrient runoff, climate change, eutrophication, fisheries related to fishing, and/or environmental changes associated with commercial presence. Further, the environmental treatment distribution techniques may operate to provide bioremediation support, such as to limit impact of effluent from certain sources (e.g., wastewater treatment plants) in certain settings (e.g., nearshore marine settings). In certain embodiments, the environmental treatment distribution techniques operate to create conditions favorable to biologic growth and/or the capture of carbon dioxide and/or other carbon-based materials, elements, and/or molecules, with or without the intent to sequester said carbon for some period of time. In certain embodiments, the environmental treatment distribution techniques operate to create an algal bloom, and/or to create conditions favorable to other aquatic and/or non-aquatic life, and/or to benefit (e.g., improve) chemical composition of the water.

In certain embodiments, the environmental treatment distribution techniques operate to distribute the silica with other materials, such as other nutrients (e.g., one or more other nutrients) and/or beneficial organisms (e.g., one or more beneficial organisms). The beneficial organisms may include phytoplankton, microalgae (e.g., diatoms and/or coccolithophorids), and/or any other suitable organisms. By way of example, the other nutrients may include, but are not limited to iron, cobalt, copper, aluminum, nitrogen, phosphorous, magnesium, manganese, calcium, sodium, potassium, and/or carbon, all in any of a variety of forms and/or solutions. For example, iron (e.g., from iron cuttings) may be distributed with the silica to strengthen activity of the organisms in the aquatic environment, so as to be in line with and/or mimic natural processes, such as volcanic ash and/or dust (e.g., from the Saharan Desert and/or China) inducing healthy algal blooms in our oceans today, for example. In certain embodiments, the other nutrients include volcanic ash and/or synthetic volcanic ash (e.g., volcanic ash in combination with one or more other nutrients or volcanic ash alone without other nutrients). The silica and/or the other nutrients may or may not be in elemental form, such as part of a different, larger, and/or more complex molecule. For example, as noted herein, the silica may be distributed with the other nutrients, such as sodium oxide, that are alkaline so as to constrain pH (e.g., resist acidification of water that may occur with introduction of silica; chemically assist with buffering and water quality).

Advantageously, the environmental treatment distribution techniques may generally benefit chemistry of water at least at distribution sites and surrounding areas, such as by facilitating a desirable balance of nutrients (e.g., ratios, such as silica to nitrogen and/or silica to phosphorus), pH, and so forth. Additionally or alternatively, the environmental treatment distribution techniques may operate to stabilize the silica cycle in certain areas (e.g., at least at the distribution sites and surrounding areas). For example, certain phytoplankton or microalgae, such as diatoms, use silicic acid (H₄SiO₄) to form silica dioxide (SiO₂), which they use in their cell walls. The environmental treatment distribution techniques may provide the silica to support the certain phytoplankton or microalgae over time (e.g., consistently over time), such as even during times without natural sources of silica and/or during times of natural low silica (e.g., relatively low silica). Additionally or alternatively, the environmental treatment distribution techniques may operate to benefit the foodweb, such as by providing building blocks for cell walls of diatoms that form a foundational and significant food source for other life (e.g., small fish and marine invertebrates). As described herein, the environmental treatment distribution techniques may be implemented by an environmental treatment distribution system that includes a silica system. The environmental treatment distribution system with the silica system is configured to contain one or more sources of silica and is also configured to efficiently, effectively, and/or dynamically process the one or more sources of silica for distribution of the silica to the aquatic environment. For example, the environmental treatment distribution system with the silica system may dynamically process the one or more sources of silica based on various factors, such as one or more characteristics of the aquatic environment (e.g., which may change over time and/or vary based on location).

The silica and/or the other nutrients may originate from different sources, either from targeted mining practices and/or byproducts of needed industrial products that are healthy, reasonably priced, and favorable to being introduced to aquatic environments (e.g., coastal marine, nearshore marine, open ocean, deep ocean and/or lakes, ponds, rivers, streams, bays, wetlands and estuaries). For example, the silica may originate from silica-rich rocks (e.g., chert; granitoid), industrial byproducts (e.g., sodium metasilicate pentahydrate), and/or other suitable source already in soluble form (e.g., sodium silicate). Further, the silica is soluble in water present in the aquatic environments, and the silica may be distributed at diluted concentrations suitable to support life in the water.

At least part of the environmental treatment distribution system may be incorporated onto one or more aquatic vessels having any of a variety of forms and/or operational features. The one or more aquatic vessels may be supported in or on water (e.g., fixed, such as to a sea floor and/or a dock; movable, such as floating and/or configured to travel through the water). The one or more aquatic vessels may be dedicated vessels for environmental treatment and/or may be vessels with other commercial purposes (e.g., fishing, deploying buoys). For example, boats, passenger ships, cruise ships, ferries, commercial vessels under charter to move people, freight, commodities, and/or other vessels may also conduct environmental treatment as disclosed herein. As used herein, the one or more aquatic vessels may also include buoys (e.g., fixed and/or floating), underwater vehicles, and so forth. The environmental treatment distribution system may be incorporated onto and/or used in conjunction with any of a variety of other aquatic vessels, maritime operating vessels, and/or aircraft, such as planes, helicopters, airborne and/or spaceborne and/or land-based vessels, equipment, containers, rail, ships, planes, drones, quadcopters, jets, rockets, balloons, and/or other devices capable of monitoring the aquatic environment and/or carrying, processing (e.g., washing, diluting, mixing, growing), and/or distributing materials using the environmental distribution system as provided herein. While certain examples show and/or describe one aquatic vessel to facilitate discussion, it should be appreciated that the aquatic vessel may include multiple aquatic vessels and/or multiple other types of vessels that may work in a coordinated manner and/or that respectively carry out certain portions of techniques described herein (e.g., one vessel that prepares and distributes silica and/or other nutrients, and one vessel that prepares and distributes organisms).

For example, the environmental treatment distribution system may include components supported on a vessel, such as an aquatic vessel. Further, the vessel may be manually controlled (e.g., by a human operator on-board the vessel), autonomously controlled (e.g., without human inputs), and/or remotely controlled (e.g., by a human operator remotely positioned away from the vessel). In particular, in certain embodiments, the vessel may be manually, autonomously, and/or remotely controlled to travel about the aquatic environment. Additionally or alternatively, the vessel and/or any portions of the environmental treatment distribution system on the vessel may be manually, autonomously, and/or remotely controlled to execute a processing program to process silica on the vessel and/or to execute a distribution program to distribute silica into the aquatic environment (e.g., at one or more locations in the aquatic environment). In certain embodiments, the vessel and/or the environmental treatment distribution system on the vessel may process and/or distribute silica while moving about the aquatic environment. However, as noted herein, at least part of the environmental treatment distribution system may be separate from and/or off-board of the vessel. For example, some or all steps to process silica may be carried out separate from and/or off-board of the vessel, and then the silica may be loaded onto the vessel for distribution into the aquatic environment.

The environmental treatment distribution system may include features to process (e.g., wash silica-rich rocks; dilute and mix into fluid) silica, such as prior to and/or while being transported at the aquatic environment. For example, a mixing system may quantify, weigh, wash, mix, move, aggregate, and/or distribute silica into the aquatic environment. The mixing system may mix the silica for dilution purposes and for solubility in the water of the aquatic environment (e.g., the silica and/or other nutrients in the fluid are bioavailable and released at similar water densities as surrounding waters when distributed into the aquatic environment; released at, in response to, and/or once a respective density is within a threshold (e.g., a target range; within 1-10 percent) of a respective density of water in the aquatic environment). Thus, the environmental treatment distribution system may be considered to store and/or begin with one or more initial silica sources (e.g., silica-rich rocks and/or industrial products in solid and/or liquid form), and the environmental treatment distribution system may process the one or more initial silica sources to generate one or more silica solutions (e.g., modified silica; adjusted silica solutions) having one or more desired parameters (e.g., target parameters) for distribution to the aquatic environment. Further, the environmental treatment distribution system may carry out the distribution to the aquatic environment.

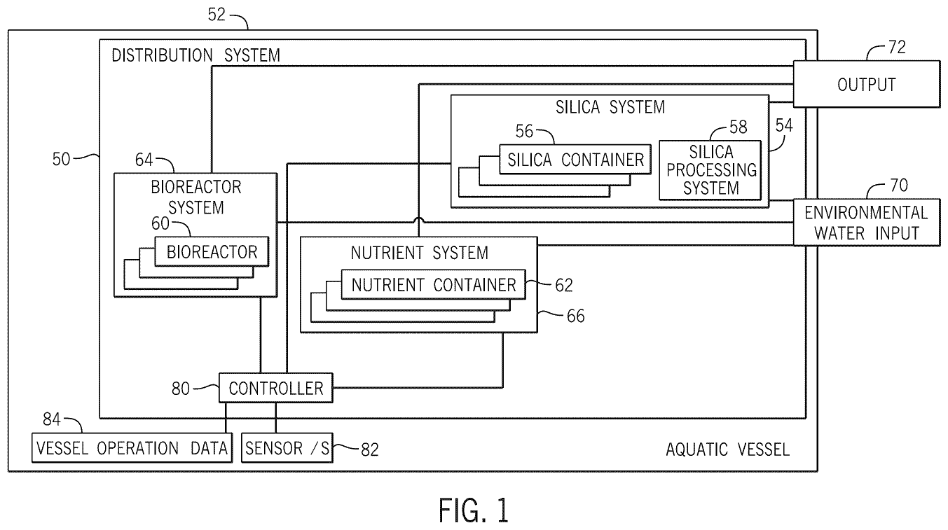

With the preceding in mind, FIG. 1 is a schematic illustration of an embodiment of an environmental treatment distribution system 50, also referred to as the distribution system 50, implemented with an aquatic vessel 52, also referred to as the vessel 52. The distribution system 50 may be, at least in part, disposed on, coupled to, attached to, placed on or in, or tethered to the vessel 52. In certain embodiments, the distribution system 50 may be arranged as part of an accessory vessel of a main vessel.

As shown, the distribution system 50 includes a silica system 54, which may include one or more silica containers 56 (e.g., storage and/or processing containers) and/or a silica processing system 58 (e.g., a silica washing system and/or a silica mixing system). The silica system 54 may include the one or more silica containers 56 to hold the silica (e.g., one or more initial silica sources). In certain embodiments, at least one of the one or more silica containers 56 may hold the silica as the silica-rich rocks, which may be in a form of powder and/or pieces of rocks. Additionally or alternatively, at least one of the one or more silica containers 56 may hold the silica as industrial byproducts, such as sodium metasilicate pentahydrate. Additionally or alternatively, at least one of the one or more silica containers 56 may hold the silica as aquaculture-related products, such as sodium silicate. Within the one or more silica containers 56, the silica may be a solid, such as powder and/or pieces without presence of a liquid; and/or the silica may be a liquid, such as dissolved into a liquid; and/or the silica may be a solid exposed to and/or held in a liquid to facilitate dissolution of the silica into the liquid over time. When the distribution system 50 utilizes silica-rich rocks as a source of silica, the silica system 54 may include the silica processing system 58 to wash (e.g., dilute) the silica-rich rocks to release silica from the silica-rich rocks and/or powders to mimic a natural process of releasing silica from silica-rich rocks in an environment albeit more efficient and effectively, such as to mimic rainwater running over the silica-rich rocks inducing chemical weathering in the environment).

In certain embodiments, a process to release the silica from the silica-rich rocks may include washing the silica-rich rocks with a washing fluid (e.g., dilution fluid), such as an acid (e.g., acetic acid, hydrochloric acid, and/or hydrofluoric acid). The silica may dissolve and/or react into the washing fluid, thereby releasing the silica from the silica-rich rocks. Further, with the silica dissolved into the washing fluid, the process may include adding a buffer (e.g., to increase pH) to the washing fluid while ensuring the silica remains water soluble. In some cases, the buffer may be added until a pH of the washing fluid (with the silica and the buffer) matches or corresponds to a pH of the water of the aquatic environment (e.g., within 2, 3, 4, 5, 10, 15, 20, or 25 percent; is greater than 6, 6.5, 7, 7.5, 8, or 8.5; is between 7 and 9, 8 and 8.5). The washing fluid with the silica and optionally the buffer may be transferred and/or collected, such as into one of the one or more silica containers 56. In certain embodiments, additional fluids (e.g., water, such as water from the aquatic environment) may be added to the washing fluid with the silica and optionally the buffer (e.g., in the one of the one or more silica containers 56; prior to distribution into the aquatic environment), such as to dilute the silica and to prepare the silica with a respective density that is the same and/or less than a respective density of the water into which the silica will be distributed (e.g., surface water, in the photic zone). Advantageously, this dilution and the relative, respective density of the silica may increase likelihood that the silica will float and not sink in the water, at least for some desirable period of time (e.g., for longer than if prepared with a respective density that is greater than a respective density of the water into which the silica will be distributed), and thus facilitates uptake of the silica by phytoplankton and/or provides desired changes in the water.

It should be appreciated that certain aspects of processing and/or mixing operations described herein may be performed manually (e.g., by a human operator), autonomously (e.g., without human inputs), and/or remotely (e.g., by a human operator remotely positioned away from controlled components). For example, a human operator may place the silica-rich rocks in at least one of the one or more silica containers 56, and a controller 80 may provide control signals to one or more flow control valves to apply the washing fluid and/or to operate an electronically-controlled agitator (e.g., air system; stirrer) to facilitate washing the washing fluid over the silica-rich rocks within the one or more silica containers 56. The controller 80 may provide the control signals based on inputs provided by the human operator (e.g., via inputs buttons, such as via virtual inputs buttons on a touchscreen display) and/or based on any other data described herein (e.g., sensor data). In any case, the distribution system 50 may process the silica-rich rocks to form the silica solution having one or more desired parameters for distribution to the aquatic environment.

To facilitate access to silica-rich rocks, it may be desirable to obtain, collect, identify, and/or filter (e.g., sort) the silica-rich rocks produced via other processes, such as silica-rich sand, powder, and/or rock pieces (e.g., chips) produced via mining, construction, and so forth. In some cases, mining may be intended to generate silica-rich sand for other purposes, such as fracking. However, the mining may generate at least some silica-rich sand with a particle size less than 0.1, 0.2, 0.3, 0.4, 0.5, 0.8, or 1.0 millimeters (mm), which may too small to be useful in fracking, but which may be useful in environmental treatment techniques described herein. Advantageously, the mining may generate large volumes of such sand with very fine particle size, which is desirable for environmental treatment techniques described herein. It is presently recognized that such sand may include variable composition (e.g., variable amounts of silica, aluminum, oxygen, iron, and other minerals). Accordingly, composition of various samples of sand from various sources may be accessed and/or evaluated, and a particular sand source (or combinations of sand sources) may be selected for storage in the one or more containers and/or for use with a particular target location of the aquatic environment (e.g., based on inputs described herein, such as one or more characteristics of the particular target location of the aquatic environment). Indeed, the particular sand source (or combinations of sand sources) may be selected and utilized for the particular target location at a first time, and another particular sand source may be selected and utilized for the particular target location at a second time (e.g., as the one or more characteristics of the particular target location may change over time). Similarly, the particular sand source (or combinations of sand sources) may be selected and utilized for the particular target location, and another particular sand source may be selected and utilized for another particular target location (e.g., as one or more characteristics of the particular target location and the another particular target location may vary from one another).

It should be appreciated that, in certain cases, the particular sand source and/or other silica source may be chosen for simpler, faster, and/or lower cost preparation techniques for streamlining preparation (e.g., as a single factor or as one of multiple factors, such as in combination with the one or more characteristics of the particular target location). For example, the distribution system 50 may identify that multiple sources are acceptable for the particular target location, and the distribution system 50 may select one source from the multiple sources based on the one source being simpler, faster, and/or lower cost than other sources of the multiple sources. As another example, the distribution system 50 may identify that one source of multiple sources is capable of forming the silica solution that most closely satisfies or meets the one or more desired parameters (e.g., silica-rich rock, such as silica-rich rock, such as silica-rock from a nearby source), but the distribution system may select another source of the multiple sources (e.g., sodium metasilicate pentahydrate or sodium silicate) due to processing being simpler, faster, and/or lower cost. This may be particularly useful when the distribution system 50 takes timing aspects into account (e.g., the distribution system 50 determines that the silica solution should be distributed at a particular time, such as within a particular time window), such as a desired time for distribution based on arrival time at a target location, forecast and/or predicted changes to the water (e.g., tides, currents), forecast and/or predicted environmental changes (e.g., incoming storms), and/or a schedule that sets forth multiple distribution times that rely on efficient preparation, and so forth. For example, the distribution system 50 may determine that washing silica-rich rocks may not produce sufficient silica by the desired time for distribution, and thus, the distribution system 50 may select a silica source that processes more quickly.

In certain embodiments, the distribution system 50 described herein may monitor (e.g., receive and/or analyze data indicative of) the one or more characteristics of the particular target location and/or may monitor available sand sources over time to identify appropriate (e.g., best, sufficient, suitable) matches for environmental treatment operations. For example, the distribution system 50 may monitor the one or more characteristics of the particular target location continuously, periodically, in response to an event (e.g., upon arrival at the particular target location; upon beginning travel to the particular target location; operator input or request), according to a schedule, and so forth. For example, the distribution system 50 may monitor the available sand sources continuously, periodically, in response to the event, according to the schedule, in response to receiving the one or more characteristics of the particular target location, and so forth. In certain embodiments, the one or more silica containers 56 may each be labeled with (e.g., associated with, such as in a lookup table of a database) one or more respective parameters, and the distribution system 50 may select one of the one or more silica containers 56 and/or determine (e.g., select or generate) the processing program based on the one or more parameters (e.g., to achieve the desired parameters for distribution).

As noted herein, the one or more silica containers 56 may include multiple silica containers 56 that hold multiple different silica compositions (e.g., types, sources, such as silica-rich rock and/or any of a variety of industrial byproducts), and as such, the distribution system 50 may monitor the one or more characteristics of the particular target location and/or may monitor available silica sources over time to identify appropriate silica sources to store in the multiple silica containers 56 and/or to select an appropriate match for distribution from one of the multiple silica containers 56. It should be appreciated that similar analysis and selection may be carried out for other silica sources described herein.

Further, silica-rick rocks or minerals may be mafic (e.g., dark colored—such as pyroxenes or amphiboles) or felsic (e.g., light colored—such as quartz, potassium feldspar, and/or plagioclase) in composition, but once weathered or pulverized an elemental breakdown separates the silica from primarily the oxygen and/or the aluminum, and secondarily from potassium, magnesium, sodium, iron II or magnesium, and/or sometimes zinc, manganese/lithium, cobalt, manganese, scandium, titanium, vanadium, and/or Iron III. In some cases, it may be desirable to utilize (e.g., only utilize) the silica-rich rocks produced within a vicinity (e.g., within 5, 10, 50, or 100 miles; a threshold vicinity) of the aquatic environment to reduce transport costs and time along with emissions, and/or to provide the silica and any other minerals released from the silica-rich rocks that would reach the aquatic environment via natural processes (e.g., via runoff due to rainwater). In certain embodiments, the one or more silica containers 56 may include multiple silica containers 56 that are each labeled with (e.g., associated with, such as in a lookup table of a database) a respective source location, and the distribution system 50 may select one of the multiple silica containers 56 based on the source location corresponding to the particular target location (e.g., being with the threshold vicinity). In certain embodiments, the distribution system 50 may select one of the multiple silica containers 56 based on the source location being closest to the particular target location (e.g., among available options; among silica sources currently in the one or more silica containers 56; the source location may be one factor, such as a highest priority factor). In certain embodiments, if there is only one silica container of the multiple silica containers 56 that includes silica with a source location that corresponds to the particular target location, the distribution system 50 may select this silica container from among the multiple silica containers 56. As another example, if two or more silica containers of the multiple silica containers 56 include silica with respective source locations that correspond to the particular target location, the distribution system 50 may select one silica container from among the two or more silica containers (e.g., based on other factors, such as supply amount, other planned distribution locations and respective suitable sources, simplicity, quickness, and/or cost, as noted herein). In certain embodiments, the distribution system 50 may only select one or more silica containers 56 that have respective source locations that correspond to the particular target location (e.g., within the threshold vicinity), and may exclude all silica containers 56 that have respective source locations that do not correspond to the particular target location (e.g., outside of the threshold vicinity). In certain embodiments, if at least one silica container of the one or more silica containers 56 includes silica with a source location that corresponds to the particular target location, the distribution system 50 will select from the at least one silica container of the one or more silica containers 56. In certain embodiments, if none of the one or more silica containers 56 includes silica with a source location that corresponds to the particular target location, the distribution system 50 will select based on other factors such as simplicity, quickness, and/or cost (e.g., to achieve the one or more desired parameters of the silica solution). Further, as noted herein, the distribution system 50 may selected based on other factors such as simplicity, quickness, and/or cost (e.g., to achieve the one or more desired parameters of the silica solution; under certain conditions, such as time constraints) even when silica with a source location that corresponds to the particular target location is available. In this way, the distribution system 50 may dynamically select the silica source(s) according to various factors, but may consider source location in an effort to mimic or represent a natural process of releasing silica from silica-rich rocks nearby the particular target location). While certain examples herein describe preparation of the silica on-board the vessel 52, it should be appreciated that at least some or all of the preparation (e.g., the washing and/or reaction of the silica-rich rocks) may be carried out on-shore and then the silica may be transferred to the one or more silica containers 56, for example.

When the distribution system 50 utilizes sodium metasilicate pentahydrate as a source of silica, the silica system 54 may include the silica processing system 58 to mix the sodium metasilicate pentahydrate with a fluid (e.g., water, such as water from an aquatic environment; mix within the one or more silica containers 56; provide the fluid into the one or more silica containers 56 to mix the sodium metasilicate pentahydrate with the fluid). It should be appreciated that certain aspects of processing and/or mixing operations described herein may be performed automatically (e.g., by the distribution system 50) and/or manually (e.g., by a human operator). For example, a human operator may provide the sodium metasilicate pentahydrate and the fluid to the one or more silica containers 56, and the silica processing system 58 may include an electronically-controlled agitator (e.g., air system; stirrer) to facilitate mixing of the sodium metasilicate pentahydrate and the fluid within the one or more silica containers 56. A mixing speed, agitator size, water flow parameters, water temperature, salinity concentrations, chlorophyll readers, density, and/or other suitable parameters prior to and/or during mixing may be input to operate the electronically-controlled agitator to facilitate mixing in a desirable manner, such as to achieve desired parameters for distribution to the aquatic environment. In certain embodiments, a flow control valve may control flow of the fluid to mix into the sodium metasilicate pentahydrate, and the distribution system 50 may automatically operate the flow control valve to adjust (e.g., enable, block, stop) flow of the fluid. However, any of these steps may be performed by the human operator, via inputs of the human operator, and/or via automatic processing and control actions by the distribution system 50 (e.g., based on the inputs and/or data, such as sensor data).

When the distribution system 50 utilizes sodium silicate as a source of silica, the silica system 54 may receive the sodium silicate as a source fluid (e.g., in water; an initial sodium silicate solution with an initial dilution or density, such as about 30 to 40 percent, 32 to 38 percent, or 33 to 35 percent sodium silicate solution). In such cases, the silica processing system 58 may further dilute the sodium silicate with fluid (e.g., additional fluid; water, such as water from the aquatic environment; mix and dilute within the one or more silica containers 56; provide the fluid into the one or more silica containers 56 to mix and dilute the sodium silicate with the fluid; a diluted sodium silicate mixture with an adjusted dilution or density). It should be appreciated that certain aspects of processing and/or mixing operations described herein may be performed automatically (e.g., by the distribution system 50) and/or manually (e.g., by a human operator). For example, a human operator may provide the sodium silicate and add the fluid to the one or more silica containers 56, and the silica processing system 58 may include the electronically-controlled agitator to facilitate mixing of the sodium silicate and the fluid within the one or more silica containers 56. In certain embodiments, a flow control valve may control flow of the fluid to mix into the sodium silicate, and the distribution system 50 may automatically operate the flow control valve to adjust (e.g., enable, block, stop) flow of the fluid. However, any of these steps may be performed by the human operator, via inputs of the human operator, and/or via automatic processing and control actions by the distribution system 50 (e.g., based on the inputs and/or data, such as sensor data).

The distribution system 50 may process the silica prior to and/or while the silica is transported at the aquatic environment (e.g., via the vessel 52). When multiple silica containers 62 are present, each may hold a same or different silica source (e.g., one with silica-rich rock from a first source and/or with a first parameter, and one with silica-rich rock from a second source and/or with a second parameter; one with silica-rich rock and one with sodium metasilicate pentahydrate; one with silica-rich rock and one with any suitable industrial byproduct, such as sodium silicate; one with sodium metasilicate pentahydrate and one with any suitable industrial byproduct, such as sodium silicate; one or more with silica-rich rock from one or more sources, one or more with sodium metasilicate pentahydrate, and/or one or more with any suitable industrial byproduct, such as sodium silicate; and/or any combination thereof). It should be appreciated that the silica-rich rock may be converted to powders (e.g., pulverization) at any suitable time of processes described herein, such as prior to being loaded onto the vessel 52 and/or prior to being transported at the aquatic environment.

In certain embodiments, the distribution system 50 also includes at least one environmental water input 70 that is arranged to transfer water or fluid from the aquatic environment (e.g., ocean water, sea water, river water) into the distribution system 50. For example, the transferred nutrient rich water from the aquatic environment and/or processed water (e.g., deionized and/or filtered via macro-or micro-filters such as through membrane modules comprised of polyvynlidine difluoride) may be provided to the silica system 54 to mix with the silica (e.g., dissolve the silica; dilute the silica).

The distribution system 50 includes at least one output 72 that is shaped and sized to transfer the silica (e.g., processed silica; modified silica; adjusted silica solution) from the one or more silica containers 56 to the aquatic environment. In certain cases, each silica container 56 is coupled to a respective dedicated output 72. In certain cases, multiple silica containers 56 may share one output 72. In one example, each output 72 includes a conduit that is fluidly coupled to at least one of the silica containers 56. With multiple outputs 72 (e.g., multiple conduits, each fluidly coupled to at least one of the silica containers 56), the multiple outputs 72 may have respective separate output locations (e.g., respective end portions are physically separated from one another) or a shared output location (e.g., connected to one another in the water). An outlet point of the at least one output 72 is positioned at or near an exterior of the vessel 52, such that silica distributed and exiting the output 72 is distributed on or in the water when the vessel 52 is operational or otherwise in the water. In certain embodiments, the at least one output 72 includes at least one end portion (e.g., where the silica exits into the water) with a floatation device to provide outflow of the silica at or near a surface of the water (e.g., within surface waters; in a photic zone that receives sunlight to support photosynthesis).

In certain embodiments, the distribution system 50 may also include one or more bioreactors 60 that promote growth of one or more organisms and/or one or more nutrient containers 62 that support one or more nutrients (e.g., other than silica). The one or more bioreactors 60 may be considered to be part of a bioreactor system 64, and the one or more nutrient containers 62 may be considered to be part of a nutrient system 66. It should be appreciated that the silica system 54, the bioreactor system 64, and the nutrient system 66 may be separate systems (e.g., distinct, physically separate components; separate hardware equipment) that are operated together to carry out techniques described herein, or may include some separate components (e.g., containers) and also some shared components (e.g., mixers, conduits, outputs) to carry out techniques described herein. The one or more bioreactors 60 may include multiple bioreactors 60, and each individual bioreactor 60 may hold and promote growth conditions for a particular organism type. In this way, the one or more bioreactors 60 may help induce biological growth for certain purposes, such as remediation purposes (e.g., removing unwanted components, such as metals and/or pollutants). In one embodiment, the distribution system 50 includes multiple bioreactors 60 holding a same type of organism. In one embodiment, respective different bioreactors 60 hold different types of organisms. An individual bioreactor 60 as provided herein may include an air supply, a nutrient-rich water intake (e.g., the environmental water input 70), an outlet (e.g., the output 72), and light source. The light source may be internal or external, depending on the characteristics of the bioreactor housing. The bioreactor housing is formed from durable materials that may be opaque (e.g., fiberglass; when light is provided from an interior) or at least partially translucent or transparent to light (e.g., polycarbonate; when light is provided from an exterior). Further, the housing may be a single-layer or multi-layer housing. In one example, bioreactor conditions may be monitored for appropriate growth via sensor/s 82 (e.g., fluorometer (measuring phycocyanin, phycoerythrin, and chlorophyll)) positioned at desired locations within the bioreactor 60.

The distributed organisms may include one or more aquatic organisms. In an embodiment, the organisms may include one or more plankton types. These plankton may be of different or similar types and/or composition (e.g., silica, carbonate, organic material), including but not limited to phytoplankton, zooplankton, autotrophs, diatoms, coccolithophorids, dinoflagellates, foraminifera, grazers, autotrophs, or otherwise. In an embodiment, the organisms include microalgae (phytoplankton) such as diatoms, foraminifera, and/or coccolithophorids. These types of plankton and/or mixes of plankton may be chosen based on visible characteristics, or genetic characteristics, or other characteristics, any combination of characteristics, or chosen by algorithm, or chosen randomly, or chosen by some other process, or not chosen at all. These types of plankton may be chosen due to a local environment for distribution and/or based on whether silica and/or carbonate would be useful compositions for growth. For example, certain types of plankton (e.g., coccolithophorids; composed of and/or produce carbonate) may be particularly desirable in nearshore marine areas and deltas, but may not be desirable (at least relative to other types of plankton) in open oceans. These plankton may be chosen due to their characteristics in single-species populations or due to their characteristics when interacting with other organisms and/or to the water column including but not limited to water movement, composition, and/or stacking of water masses.

Distributed organisms may include plankton and/other organisms created with biotechnology, genetic modification, gene editing, or other techniques that enable the change, creation, limitation, and/or increase in single or multiple traits, characteristics, behaviors, activities, growth rates, compositions, sinking rates, nutrient uptake, nutrient release, and/or interaction with other biologics or non-biologics throughout the water column. Distributed organisms may include organisms intentionally bred for specific characteristics, and/or species created and/or modified using processes which can alter their characteristics, genome, or other factors, such as but not limited to bioengineering, genetic modification, or gene editing. By way of example, the one or more nutrient containers 62 may hold the one or more nutrients such as, but are not limited to iron, cobalt, copper, aluminum, nitrogen, phosphorous, magnesium, manganese, calcium, sodium, potassium, zinc, barium, boron, cadmium, lead, mercury, nickel, selenium, silver, and carbon and/or polycyclic aromatic hydrocarbons, and dioxins/furans, each in any suitable form and/or solution. In an embodiment, the other nutrients include volcanic ash or synthetic volcanic ash (e.g., volcanic ash in combination with one or more other nutrients or volcanic ash alone without other nutrients) or dust similar to what goes airborne from the Saharan Desert and/or China.

The distribution system 50 may operate to selectively distribute the silica separately. Indeed, the distribution system 50 may be configured to distribute only the silica (e.g., may be devoid of the one or more bioreactors 60 and the one or more nutrient containers 62). However, in certain embodiments, the distribution system 50 may be configured to distribute the silica with the organisms and/or the other nutrients (e.g., one or more organisms and/or one or more other nutrients; together, such as simultaneously (e.g., at a same time), sequentially (e.g., at different times; one after another), at overlapping times, and/or in a coordinated manner). Additionally, the distribution system 50 may be configured to distribute the silica with the organisms and/or the other nutrients according to certain parameters (e.g., timing, paths, ratios; a dispersal program). For example, the distribution system 50 may determine and/or receive the dispersal program, which includes respective rates and/or respective times for distribution of the silica, the organisms, and/or the other nutrients to achieve desired respective concentrations (e.g., absolute and/or relative concentrations; determined based on one or more factors described herein and intended to facilitate health algal blooms) in the water. For example, the dispersal program may include distribution of the silica, the organisms, and the nutrients at desired respective rates and/or over respective times to provide a substantially continuous flow of the silica, the organisms, and the nutrients into the water over time during one dispersal sequence or event (e.g., without gaps; without programmed or intentional gaps; without substantial gaps, such only with gaps of less than 5, 4, 3, 2, 1, or 0.5 minutes). For example, the distribution system 50 may release the silica and any iron at separate times and/or via separate outputs 72 (e.g., alongside one another; in proximity to one another). Additionally or alternatively, the distribution system 50 may release the silica and any iron with a target ratio (e.g., more silica than iron, such as 5 to 1 or 10 to 1 or 20 to 1 or 25 to 1). The distribution system 50 may operate in a manner to prepare and disperse nutrients in a manner that is in line with and/or follows typical concentrations and certain elements that are known to be present in natural rocks in an environment, as well as that are known to facilitate healthy algal blooms. For example, silica, aluminum, and iron are major constituents of volcanic ash, whereas calcium and magnesium are minor constituents of volcanic ash. As another example, dust, silica, potassium, magnesium, and carbonate (e.g., calcite/dolomite) are major constituents of dust, whereas iron and aluminum are minor constituents of dust. In certain embodiments, the distribution system 50 may disperse the silica and the other nutrients such that silica is a most abundant element dispersed into the water. Notably, silica is a main component of both volcanic ash and dust (e.g., the main component; approximately 70 percent, and can be up to 100 percent in cases of chert from crystalline basement material). As such, the distribution system 50 may provide benefits within the water in a manner that is based on and in line with natural processes and natural materials.

In certain embodiments, the distribution system 50 may release the silica and the organisms simultaneously or at least at overlapping times and/or via separate outputs 72 (e.g., alongside one another; in proximity to one another). In certain embodiments, the distribution system 50 may distribute the silica with multiple different types of organisms (e.g., simultaneously, sequentially, at overlapping times, and/or in a coordinated manner; at one target location or region; as part of one schedule treatment), as this is in line with and/or matches natural phytoplankton assemblages in our oceans, which typically include multiple species. For example, along with the silica, the distribution system 50 may distribute a first type of phytoplankton over a first time period, then a second type or phytoplankton over a second time period, then the first type of phytoplankton over a third time period, then the second type of phytoplankton over a fourth time period, and so on in an effort to strength the overall phytoplankton assemblage. In certain embodiments, additional steps and/or coordination may be desirable, such as to provide respective dedicated mixing systems (e.g., agitators, containers) for the silica, the organisms, and/or the other nutrients and/or respective dedicated mixing times (e.g., in cases of a shared mixing system) for the silica, the organisms, and/or the other nutrients. Such configurations and/or operations may avoid mixing the silica and the organisms together with any mixing system, and thus, may support growth and survival of the organisms during mixing and/or release processes. Further, at least for diatoms, a buffer may be provided to the water at the target location prior to and/or during addition of the organisms to support growth and survival of the organisms during the release and/or thereafter.

In any case, the distribution system 50 may select materials (e.g., the silica, the organisms, and/or the other nutrients) for distribution and/or determine timing for distribution based on one or more factors, such as a target location for release of the materials, one or more characteristics of water of the aquatic environment (e.g., at the target location for release of the materials and/or other location of the aquatic environment that is expected to be impacted by the release of the materials), one or more environmental conditions at the aquatic environment, one or more parameters of the materials (e.g., level of solubility, density, source location, available volume), and so forth. For example, the target location and/or the other location may include coastal marine, nearshore marine, open ocean, deep ocean and/or lakes, ponds, rivers, streams, bays, wetlands and estuaries to allow for silica-rich phytoplankton (e.g., diatoms) to thrive. As an example, the one or more characteristics of the water of the aquatic environment may include a flow rate, composition (e.g., nutrient concentration, including silica concentration; nutrient ratios, such as silica to nitrogen and/or silica to phosphorus), and/or types of organisms (e.g., plankton) present in the water of the aquatic environment (e.g., at the location). In certain embodiments, a pH (e.g., alkalinity) of the water of the aquatic environment is not considered (e.g., data indicative of pH is not collected and/or is not utilized to select the materials for distribution and/or determine timing for distribution). However, the pH of the water of the aquatic environment may be considered at least in order apply an appropriate buffer to or for the silica (e.g., in solution—sodium oxide being one such example) to correspond to the pH of the water of the aquatic environment prior to release of the silica into the water of the aquatic environment. For example, the distribution system 50 may monitor (e.g., continuously, periodically, in response to an event, and/or according to a schedule) the materials in the one or more silica containers 56 and/or the one or more characteristics of the aquatic environment (e.g., including environmental conditions, such as wind) to generate (e.g., initiate processing and/or further process) and/or select (e.g., already processed) the materials that are appropriate for distribution to the target location of the aquatic environment (e.g., using one or more algorithms or lookup tables; expected to produce a desired result; have corresponding or matching density, source location, pH, and/or other parameters).

The distribution system 50 includes a controller 80 (e.g., electronic controller) that controls some or all operations of components of the distribution system 50. In certain embodiments, the controller 80 controls operation of the silica system 54, including some or all components of the silica system 54. For example, the controller 80 may control the silica processing system 58. In an embodiment, the controller 80 controls operation of the one or more bioreactors 60 and/or the one or more nutrient containers 62, including some or all components thereof. For example, the controller 80 may control light, temperature, and/or other conditions at the one or more bioreactors 60 and/or the one or more nutrient containers 62. Additionally or alternatively, the controller 80 may control activation and cessation of distribution from the one or more silica containers 56, the one or more bioreactors 60, and/or the one or more nutrient containers 62. The controller 80 also controls selection of individual silica containers 56, individual bioreactors 60, and/or individual nutrient containers 62. In an embodiment, the controller 80 may be configured to provide information and/or control instructions to the vessel 52 (e.g., to provide coordinates of a target location to a vessel controller, such as to enable the vessel controller to initiate autonomous travel of the vessel 52 and/or for display to a human operator of the vessel 52; to cause the vessel 52 to autonomously travel to the target location).

The controller 80 may receive various types of data and operate responsive to the various types of data, as generally discussed herein. For example, the various types of data may include the one or more characteristics of the aquatic environment (e.g., including current and forecast environmental conditions at the aquatic environment, such as weather conditions at the aquatic environment), the one or more parameters of available silica sources (e.g., in the one or more silica containers 56; type, location, volume, density, pH), information related to available organisms (e.g., in the one or more bioreactors 60; type, growth rate, stage, volume, and so forth), information related to available nutrients (e.g., in the one or more nutrient containers 62; type, volume, density, and so forth), information related to the vessel 52 (e.g., planned route, remaining fuel), a schedule, one or more inputs from the human operator, and/or any other data described herein. As described herein, the various types of data may include sensor data, including real-time sensor data (e.g., real-time or substantially real-time).

The controller 80 may access (e.g., store and/or retrieve) one or more algorithms (e.g., machine-learning algorithms) to process the various types of data and operate response to the various types of data, as generally discussed herein. For example, the controller 80 may input some of all of the various types of data to identify a particular target location for treatment and/or to generate a processing program and/or a distribution program for the particular target location. For example, the processing program may establish the desired parameters for the silica, as well as the silica source that should be selected and the processing steps that should be performed to transition the silica source into modified silica with the desired parameters. Additionally or alternatively, the distribution program may establish a schedule, such as a timing for distribution of the modified silica, the organisms, and/or the other nutrients in a coordinated manner that is expected to result in and/or achieved desired results at the particular target location and/or other locations that may be affected by distribution at the particular target location.

As noted herein, the controller 80 may receive various types of sensor data, including sensor data from one or more sensors 82. The sensor/s 82 may include location sensors to generate location data indicative of a location of the vessel 52, water monitoring sensors to generate characteristic data indicative of one or more characteristics of the water at the aquatic environment, and/or environmental sensors to generate environmental condition data indicative of one or more environmental conditions as the aquatic environment. For example, the environmental sensors may include one or more anemometer to monitor wind direction and speed, hyperspectral cameras to monitor sediment composition and/or sinking rates, electromagnetic sensors for current readings, sonar and/or echo sounder to monitor a mudline in three-dimensions to facilitate measurement of sediment volume and/or carbon content (e.g., for total carbon delivery to the sediment), and so forth, supplemented by water and/or mud samples. For example, the sensor data may indicate location, time of year, season, water depth, air or water temperature and pressure, wind direction and speed, water current direction and speed, salinity, humidity, turbidity, pH, pCO2, light, oxygen density, dissolved oxygen, conductivity, temperature, pressure, currents velocities and directions, carbon dioxide, eDNA, types of organisms, anemometer, sediment composition and sinking rates, sediment volume, carbon content, total carbon delivery to the sediment, water column composition (e.g., including life), depth/altitude, nutrients, nutrient concentration (including silica concentration), nutrient composition and ratios (such as silica to nitrogen and/or silica to phosphorus, presence of nitrates, phosphates, ammonia plus more), radiance and irradiance, fisheries echosounder, cameras (IR, VIS), sedimentation, hydrophone array, fluorometer (measuring phycocyanin, phycoerythrin, and chlorophyll), and chlorophyll concentrations.

The sensor/s 82 may be positioned on, in, and/or below the vessel 52, on or in the distribution system 50 (e.g., coupled to the one or more silica containers 56, the one or more bioreactors 60, and/or the one or more nutrient containers 62). In certain embodiments, the sensor/s 82 may be coupled to a user device, such as a mobile device carried by a human operator associated with the distribution system 50 and/or the vessel 52. Additionally or alternatively, the sensor/s 82 may be coupled to some other vessel that is separate from the vessel 52. For example, the other vessel may include one or more other aquatic vessel, such as passenger ships, cruise ships, ferries, commercial vessels under charter, buoy (e.g., free floating and/or in-situ), underwater vehicle, aircraft, airborne and/or spaceborne and/or land-based vessels, and so forth. The other vessel may be manually controlled (e.g., by a human operator on-board the vessel), autonomously controlled (e.g., without human inputs), and/or remotely controlled (e.g., by a human operator remotely positioned away from the vessel). The other vessel may be part of the distribution system 50 and/or communicatively coupled to the distribution system 50. The other vessel may be manually controlled (e.g., by a human operator on-board the other vessel), autonomously controlled (e.g., without human inputs), and/or remotely controlled (e.g., by a human operator remotely positioned away from the other vessel).

It should be appreciated that the distribution system 50 may additionally or alternatively receive certain types of data (e.g., the location data, the characteristic data, the environmental condition data) from one or more external data sources, such as a weather service. In addition, the controller 80 may receive vessel operation data 84 (e.g., vessel speed, vessel location, vessel direction, estimated route) that is used as an input to control operations of the distribution system 50. As provided herein, the distribution system 50 may receive vessel operation data 84, may control operations of the vessel 52, and/or may provide input to a separate controller of the vessel 52.

The sensor/s 82 may monitor the one or more characteristics of the water at the aquatic environment prior to distribution of the silica, during the distribution of the silica, and/or after the distribution of the silica (and any other materials distributed by the distribution system 50). In certain embodiments, the sensor/s 82 may monitor the one or more characteristics of the water inside and/or outside an area of interest (e.g., outside the target location; outside of an area in which change is expected after treatment described herein). The controller 80 may receive the data from the sensor/s 82 and may evaluate the one or more characteristics of the water, including trends in the one or more characteristics of the water over time. The controller 80 may identify certain features prior to distribution of the silica (e.g., in the one or more characteristics and/or in the trends), such as seasonal changes in nitrogen concentration and/or seasonal changes in a silica to nitrogen ratio and/or phosphorous. Accordingly, over time, the controller 80 may predict certain changes and plan (e.g., determine, set) future distributions of the silica (e.g., amount of silica, frequency or timing of distribution, location of distribution, other nutrients) based on certain features (e.g., seasonal changes, winds, wave heights). Similarly, the controller 80 may identify certain features after the distribution of the silica (e.g., in the one or more characteristics and/or in the trends), such as seasonal changes in growth of organisms after distribution of a certain quantity of silica. Accordingly, over time, the controller 80 may predict certain changes and plan future distributions of the silica (e.g., amount of silica, frequency or timing of distribution, location of distribution, other nutrients) based on certain features (e.g., seasonal changes). Similar steps to identify, predict, and plan based on monitored changes in presence of certain flow rates, air temperatures, water temperatures, and/or other characteristics and/or environmental conditions described herein are envisioned. In this way, the distribution system 50 may dynamically adapt to process and distribute the silica in a manner that provides efficient and effective environmental treatment techniques.

The controller 80 may include at least one processor, memory, or any of a variety of other components, such as input/output interface, and a display that enable the controller 80 to carry out the techniques described herein. In addition, in certain embodiments, the controller 80 may include communication circuitry to facilitate communication with other control systems of the vessel 52, with a remote server, and/or with other members of a vessel fleet. In certain embodiments, the communication circuitry may be configured to facilitate wireless communication and/or wired communication, including but not limited to real-time data acquisition.

The processor may be any suitable type of computer processor or microprocessor capable of executing computer-executable code. In certain embodiments, the processor may also include multiple processors that may perform the operations described herein, and certain operations may be distributed between the processor and one or more remote servers. The memory may be any suitable articles of manufacture that can serve as media to store processor-executable code, data, or the like. These articles of manufacture may represent computer-readable media (e.g., any suitable form of memory or storage) that may store the processor-executable code used by the processor to perform the presently disclosed techniques. The memory may also be used to store data, various other software applications, and the like. The memory may represent non-transitory computer-readable media (e.g., any suitable form of memory or storage) that may store the processor-executable code used by the processor to perform various techniques described herein.

As noted herein, the controller 80 may use machine learning (e.g., artificial intelligence and/or deep learning) to model and recognize patterns to make decisions and/or better improve predictability of aquatic conditions and/or generate control algorithms for selection, processing, and/or distribution of materials. As used herein, machine learning refers to algorithms and statistical models that may be used to perform a specific task without using explicit instructions, relying instead on patterns and inference. In particular, machine learning generates a mathematical model based on data (e.g., sample or training data, historical data) in order to make predictions or decisions without being explicitly programmed to perform the task. In particular, as data is collected, patterns of events may emerge. The patterns may be referenced and used to predict expected events in aquatic environments especially near and in the ocean (e.g., future events; before occurrence of the events).

In some embodiments, such as during availability of particular known examples that correlate to future predictions (e.g., the set of historical data), supervised machine learning may be implemented. In supervised machine learning, the mathematical model of a set of data contains both the inputs and the desired outputs. This data is referred to as “training data” and is essentially a set of training examples. Each training example has one or more inputs and the desired output, also known as a supervisory signal. In the mathematical model, each training example is represented by an array or vector, sometimes called a feature vector, and the training data is represented by a matrix. Through iterative optimization of an objective function, supervised learning algorithms learn a function that can be used to predict the output associated with new inputs. An optimal function will allow the algorithm to correctly determine the output for inputs that were not a part of the training data. An algorithm that improves the accuracy of its outputs or predictions over time is said to have learned to perform that task. Supervised learning algorithms include classification and regression. Classification algorithms are used when the outputs are restricted to a limited set of values, and regression algorithms are used when the outputs may have any numerical value within a range.

Additionally and/or alternatively, in some situations, it may be beneficial to utilize unsupervised learning (e.g., when particular output types are not known). Unsupervised learning algorithms take a set of data that contains only inputs and find structure in the data, such as grouping or clustering of data points. The algorithms, therefore, learn from test data that has not been labeled, classified, or categorized. Instead of responding to feedback, unsupervised learning algorithms identify commonalities in the data and react based on the presence or absence of such commonalities in each new piece of data. In any case, machine learning may be used to identify the expected events.

Thus, in the distribution system 50, a predictive model may be generated (e.g., by a processor of controller 80) and stored (e.g., in a memory device accessible by the processor of the controller 80), and the predictive model may be utilized to determine (e.g., predict) appropriate distribution parameters for the target location, as described herein. For example, the predictive model may receive a variety of inputs including but not limited to the sensor data described herein, and use the variety of inputs to determine the appropriate distribution parameters including type of nutrients (e.g., silica and/or the other nutrients) and/or organisms and their source, combinations, mixing process, dilution, and/or distribution technique (e.g., timing, shared or separate outlets 72) for the target location, as described herein. The predictive model may also be utilized to predict future aquatic conditions at the target location for distribution at a future time (e.g., at a time of distribution; an arrival time, such as based on a schedule and/or current travel of the vessel 52), which in turn be input to the predictive model to determine the appropriate distribution parameters. Such predictions may enable preparation the nutrients and/or organisms in advance so that the nutrients and/or organisms are ready for distribution at the future time. The predictive model may periodically and/or continuously update based on receipt of new data (e.g., the sensor data, such as before and after treatment; utilized as training data).

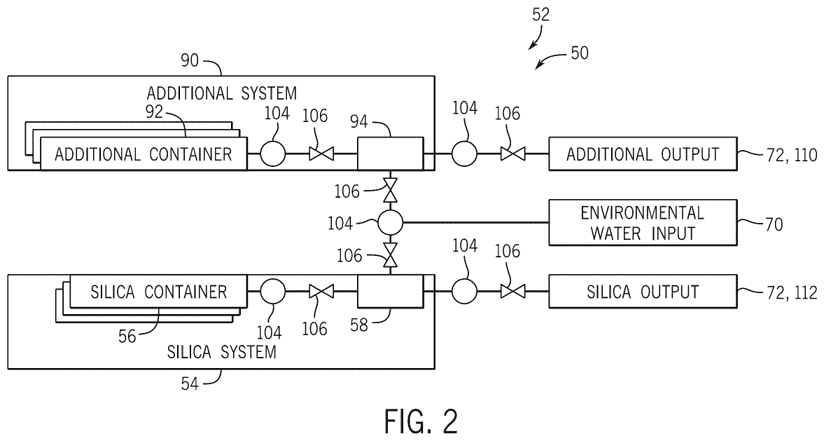

FIG. 2 is a schematic illustration of an embodiment of an arrangement of components of the distribution system 50. As shown, the silica system 54 may include the one or more silica containers 56 and the silica processing system 58. The silica system 54 may be used together with an additional system 90, which may be utilized to hold and/or process additional materials, such as organisms and/or other nutrients, as set forth with respect to FIG. 1. The additional system 90 may represent the bioreactor system 64 and/or the nutrient system 66 of FIG. 1, and further may include one or more additional containers 92 (e.g., the one or more bioreactors 60 of FIG. 1 and/or the one or more nutrient containers 62 of FIG. 1) and a processing system 94 (e.g., mixer).

One or more pumps 104 and valves 106 can be activated to fluidly couple and/or uncouple components of the distribution system 50, as shown by way of example in FIG. 2. However, other arrangements are also contemplated, and additional or fewer pumps 104 and valves 106 may be incorporated. In the illustrated arrangement, the one or more pumps 104 and valves 106 control entry of water from the environmental water input 70 to the silica system 54 and/or the additional system 90. After processing (e.g., mixing), mixed compositions (e.g., the water and silica; the water and organisms; the water and nutrients) can be distributed on or in the water via the at least one output 72, which may include multiple dedicated outputs 110, 112. It should be understood that at least one output 72 may include at least one shared output 72. Further, the distribution system 50 may utilize dedicated outputs 110, 112 and/or at least one shared output 72 based on an arrangement of the distribution system 50, the vessel 52, materials being distributed, a targeted location, and/or active weather patterns including but not limited to rainfall, currents, and wave action including but not limited to wave heights and direction, for example.

It is presently recognized that certain parameters may be particularly desirable for preparing and/or distributing the silica. For example, the distribution system 50 may calculate and/or receive a desired concentration of silica in the water, which may be determined (e.g., predicted; intended) to support healthy algal blooms. In certain embodiments, the desired concentration of silica in the water may bring a respective concentration silica closer to and/or approximately equal to a respective concentration of nitrogen (e.g., within 1, 2, or 3 percent). In any case, the distribution system 50 may calculate and/or receive a desired change to a current concentration of silica in the water to reach the desired concentration of silica in the water. In certain embodiments and under certain conditions, the desired change may be an increase of approximately 0.2 to 1.0, 0.3 to 0.8, 0.4 to 0.5 milligrams/liter. Further, the distribution system 50 may calculate and/or receive a rate of distribution, which may be determined (e.g., predicted; intended) to provide the desired concentration of silica in the water. Then, the distribution system 50 may operate to release the silica at the rate of distribution. In certain embodiments, the distribution system 50 may monitor the one or more characteristics of the water during the release of the silica, and the distribution system 50 may calculate and/or receive an adjusted rate of distribution and operate the release the silica at the adjusted rate of distribution to provide dynamic distribution over time.

The containers 56, 92 and/or the processing systems 58, 94 may include one or more stirrers, spinners, paddles, blades, pumps, and/or other features that facilitate mixing to achieve desired characteristics of the materials. For example, silica in the one or more silica containers 56 may be provided as a dry composition, such as a dry powder or sand. If distributed directly into the water in powder or sand form, the silica may not effectively disperse. Thus, mixing the silica with the environmental water (or other fluid) before distribution can enhance dispersal of the silica by allowing the silica to go into solution. The controller 80 may control the activation of mixing features by turning on mixing features, e.g., by activating a paddle, activating a pump to create turbulence to enhance mixing, or by agitating or turning the mixers. The controller 80 may also select mixing settings depending on the materials. In certain embodiments, the mixers may include a lid that may be selectively opened and closed (e.g., manually and/or via automated control).

While the processing systems 58, 94 are shown separately from the containers 56, 92 to facilitate discussion, it should be appreciated that the distribution system 50 may include these components in any of a variety of arrangements with corresponding pumps 104 and valves 106 to prepare and disperse materials as described herein. For example, each silica container 56 may have a respective silica processing system 58, a group of two or more silica containers 56 may share one silica processing system 58, and/or all silica containers 56 may share one silica processing system 58. Further, parts of the processing system 58 may be distributed and/or separated from one another. For example, washing equipment may be provided at one or more silica containers 56 that contain solid rock pieces, and the washing equipment may wash the solid rock pieces within the one or more silica containers 56. Thereafter, silica washed from the rocks may be further processed (e.g., buffer added; water added) within the one or more silica containers 56, or silica washed from the rocks may be carried in solution to other equipment for further processing and eventually distribution. As another example, at least some solid rock pieces may be transferred (e.g., by a conveyor, robotic arm, manually by a human operator) as solid rock pieces to the processing system 58 for processing (e.g., washing, dilution, mixing). As another example, mixing equipment may be provided at one or more silica containers 56 that contain silica in powder form, and the mixing equipment may mix the powder with fluid (e.g., water) within the one or more silica containers 56. Thereafter, at least a portion of this silica solution may be transferred to other equipment for further processing and eventually distribution. As another example, at least some powder may be transferred (e.g., by a conveyor, robotic arm, manually by a human operator) in powder form to the processing system 58 for processing (e.g., dilution, mixing). As another example, at least a portion of an initial silica solution may be transferred to other equipment for processing (e.g., mixing, dilution). Similar variations are envisioned for the additional system 90, such as with respect to locations of processing equipment and stages of processing (e.g., in the additional containers 92 and/or at separate equipment).

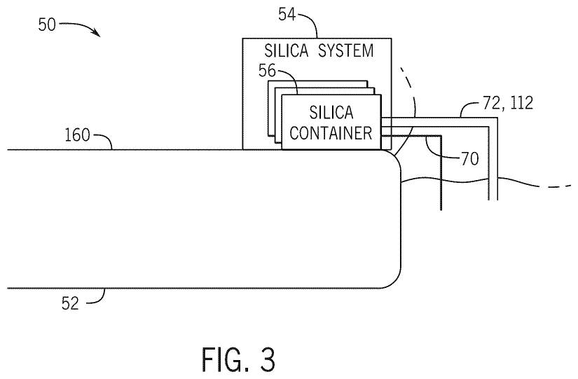

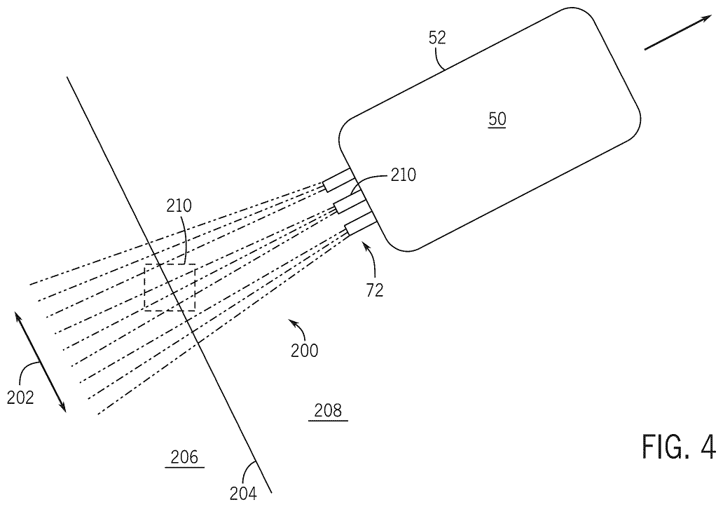

FIG. 3 is a schematic illustration of an embodiment of the vessel 52 including the distribution system 50 that incorporates the one or more silica containers 56 (and/or other components of the silica system 54, such as the silica processing system 58 of FIG. 1). As illustrated, the silica system 54 may be arranged such that the at least one environmental water input 70 is below the water line. Further, the silica system 54 may be arranged such that the at least one output 72 is positioned to be at or near (e.g., above and/or below) an estimated water line location on the vessel 52. For larger aquatic vessels, one or more components of the distribution system 50 may be located in an interior space 150. Smaller or near-shore vessels, as illustrated in FIG. 5, may be arranged with at least certain components of the distribution system 50, such as the silica system 54, arranged on a vessel deck 160.

FIG. 4 shows an example dispersal pattern of distributed materials 200 (e.g., silica, such as silica dissolved in water and without addition of other nutrients and/or organisms; silica and other nutrients; silica and organisms; silica, other nutrients, and organisms). With the distribution system 50 incorporated as part of the vessel 52, the distributed materials 200 are distributed via the at least one output 72 of the distribution system 50, such that the distributed materials are generally distributed onto or near a water surface of the aquatic environment. In certain embodiments, the desired distribution may be at a depth deeper than the water surface of the aquatic environment but within the photic zone.

The aquatic environment may include coastal marine, nearshore marine, open ocean, deep ocean and/or lakes, ponds, rivers, streams, bays, wetlands and estuaries, for example. The aquatic environment may have any suitable depth, such as a depth of approximately over 100, less than 10, 10 to 100, 20 to 70, or 30 to 50 meters. In any case, dispersal techniques represent the movement, spread and/or transport of the silica, the other nutrients, and/or the organisms introduced into aquatic environments following dispersal vectors such as abiotic vectors (e.g., ocean currents, wind, waves). Dispersal patterns (e.g., spread or width 202 across the vessel 52; angle relative to the water surface; dispersal density, such as from multiple outputs 72 or fewer outputs, such as every other output 72 across the vessel 52) of the distributed materials 200 may be determined (e.g., selected, adjusted) and implemented (e.g., via the at least one output 72) based on one or more factors, the target location, the one or more characteristics of water of the aquatic environment (e.g., at the target location for release of the materials and/or other location of the aquatic environment that is expected to be impacted by the release of the materials), the one or more environmental conditions at the aquatic environment, and so forth. As shown, the at least one output 72 includes multiple outputs (e.g., three outputs, although any number of outputs are envisioned). In certain embodiments, all of the multiple outputs 72 may distribute silica during one time period, all of the multiple outputs 72 may distribute the other nutrients over another time period, and all of the multiple outputs 72 may distribute the organisms over another time period (e.g., sequentially, such as to provide continuous flow). In certain embodiments, one or more outputs of the multiple outputs 72 may be dedicated outputs to distribute the silica, one or more other outputs of the multiple outputs 72 may be dedicated outputs to distribute the other nutrients (e.g., all of the nutrients, or dedicated output(s) for each type of nutrient), and one or more other outputs of the multiple outputs 72 may be dedicated outputs to distribute the organisms (e.g., all of the organisms, or dedicated output(s) for each type of organism). In such cases, the multiple outputs 72 may distribute the silica, the other nutrients, and/or the organisms simultaneously, sequentially (e.g., such as to provide continuous flow), at overlapping times, and/or in some other coordinated manner.

A target location for treatment may have any suitable size, such as an area of approximately over 100, less than 1, 1 to 100, 2 to 50, 3 to 25, 4 to 20, 5 to 15, or 6 to 10 square kilometers. It should be appreciated that a desired distribution location(s) within the area may vary based on the one or more factors. For example, the desired distribution location(s) within the area may be at or proximate to a center of the area, particularly if the area is landlocked, substantially landlocked, has low waves and/or current (e.g., below a threshold), and/or other stable characteristics. Further, the vessel 52 may carry out one or more dispersals while stationary at a position in the area (e.g., at only one desired distribution location within the area, such as the center of the area), multiple dispersals while stationary at multiple positions in the area, and/or one or more dispersals while moving through the area.

In certain embodiments, the desired distribution location(s) may be selected based on presence of sensors in the water. For example, the distribution system 50 may communicate with and/or include measurement, reporting, verification (MRV) equipment 210 in the water, such as at or near a surface and/or at or near a floor of the aquatic environment. The MRV equipment 210 may be carried with the vessel 52 and/or deployed separately in the aquatic environment (e.g., supported by a buoy). The MRV equipment 210 may include and/or overlap with the sensor/s 82 described with reference to FIG. 1. However, generally, the MRV equipment 210 may include sensors to monitor characteristics of the aquatic environment before, during, and/or after the distribution of the silica, the organisms, and/or the other nutrients. Thus, the MRV equipment 210 may generate data that is indicative of and can be used to evaluate effects of the distribution of the silica, the organisms, and/or the other nutrients. The MRV equipment 210 may provide data indicative of carbon dioxide, photosynthetically active radiation, dissolved oxygen, optical sediment traps, chlorophyll, conductivity, temperature, and/or other parameters. The MRV equipment 210 and/or other operations may be implemented to collect and evaluate water samples and/or mud samples from the floor of the aquatic environment, such before, during, and/or after the distribution of the silica, the organisms, and/or the other nutrients. For example, the water samples indicate water composition, environmental DNA (e.g., phytoplankton identification), and/or other parameters, while the mud samples indicate mineralogy, carbon content, and environmental DNA (e.g., sunk phytoplankton identification; preserved in the mud). It should be appreciated that this information may be used to dynamically adjust dispersal (e.g., in real-time), adjust future dispersals (e.g., for the target location and/or other target locations), train models (e.g., machine learning models) that are used to determine preparation and dispersal programs, and so forth.

In certain embodiments, it may be desirable to disperse the materials at or proximate to an intersection 204 (e.g., boundary) between a tributary 206 (e.g., river) and ocean water 208 (e.g., within a distance range of between about 1 to 20, 2 to 10 miles, or between 3 to 8 kilometers (km) at or proximate to where the tributary 206 meets and mixes with the ocean water 208), depending on local currents and movement of water, as well as at relatively shallow depths in areas where surface currents are relatively lower (e.g., compared to relatively deep depths) or in areas with very low horizontal movement of water. Further, a depth at the intersection 204 may be less than or approximately equal to about 100, 200, or 300 meters. In certain embodiments, it may be desirable to disperse the materials at or proximate to the target location, such as the intersection 204, that has a depth less than or approximately equal to about 100, 200, or 300 meters. The distribution system 50 may be configured to receive and process the various types of data described herein, such as data indicative of the currents of the water in the aquatic environment, to determine (e.g., using one or more algorithms, such as machine learning algorithms) a particular location for distribution (e.g., relative to the tributary 206 and/or the ocean water 208), as well as to then select the silica source(s), generate the processing program, and/or generate the distribution program based on the particular location for distribution.

Without operation of the distribution system 50 described herein, a change in silica concentration may be observed across the intersection 204. For example, without operation of the distribution system 50 described herein, in the tributary 206, the silica may have a first concentration, and the silica may have one or more first ratios relative to one or more other nutrients (e.g., silica to nitrogen, silica to phosphorus). Further, without operation of the distribution system 50 described herein, in the ocean water 208 the silica may have a second concentration that is different than the first concentration, and the silica may have one or more second ratios relative to one or more other nutrients (e.g., silica to nitrogen, silica to phosphorus) that is different than the one or more first ratios. Indeed, in certain cases without operation of the distribution system 50 described herein, the first concentration is greater than the second concentration, which natural processes can also augment (as expected) such as with storms and weather. Advantageously, the distribution system 50 takes these natural processes into account. Accordingly, providing the distributed materials 200 at or proximate to the intersection 204 at or over which such silica concentration changes (e.g., drops off as water flows from the tributary 206 to the ocean water 208) may maintain, replenish, or provide supportive amounts of silica to facilitate growth of organisms at or proximate to the intersection 204. Advantageously, by targeting the intersection 204, the distribution system 50 may provide impact to growth of organisms at or proximate to the intersection 204, and the organisms may proliferate to provide environmental treatment at or proximate to the intersection 204. Further, the organisms may move into the ocean water 208 to provide environmental treatment at or within the ocean water 208. In this way, the distribution system 50 may provide efficient and effective environmental treatment across these regions with relatively low amount of distributed materials 200 (e.g., as compared to an amount of distributed materials 200 that may be needed to provide environmental treatment in open ocean waters). In certain embodiments, for a particular target location, the distribution system 50 may disperse between about 1 to 10, 2 to 8, or 3 to 5 tonnes per dispersal and/or greater than about 1, 2, 3, 4, 5, 6, 7, 8, 9, or 10 tonnes (e.g., per release; per period of continuous output; pulverized equivalent rock or powder, formed primarily of silica). Additionally, for a particular target location, the distribution system 50 may carry out between about 1 to 10, 2 to 8, 3 to 6, or 1 to 2 dispersals per day for some period of time and/or at least 1, 2, 3, 4, 5, 6, 7, 8, 9, or 10 dispersals per day for some period of time. As one non-limiting example, on an annual basis for a particular target location, the distribution system 50 may disperse between about 10 to 2500, 30 to 1800, or 50 to 1000 tonnes per year.

In certain embodiments, the distribution system 50 may provide automatic shutoff or deactivation of distribution. Termination of distribution may be based on inputs by a human operator and/or based on the various types of data described herein, such as sensor data, for example. For example, the sensor data may be indicative of vessel movement out of a geographic location of interest. Other examples include automated processing of data (signals such as wave data, oceanographic data, water sampling data, environmental data, or other data that indicate dispersion should be stopped). In an embodiment, the environmental treatment distribution system may include control algorithms that increase, decrease (e.g., to non-zero), stop (e.g., to zero), and/or start dispersion operations based on one or more factors, as described herein.

While only certain features have been illustrated and described herein, many modifications and changes will occur to those skilled in the art. It is, therefore, to be understood that the appended claims are intended to cover all such modifications and changes as fall within the true spirit of the disclosure. Any features shown or described with reference to FIGS. 1-4 may be combined in any suitable manner, and systems and components described with reference to FIGS. 1-4 may be operated (e.g., at least in part by the controller 80) to perform methods to carry out any techniques described herein.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. § 112(f). However, for any claims containing elements designated in any other manner, it is intended that such elements are not to be interpreted under 35 U.S.C. § 112(f).

1. An environmental treatment distribution system, comprising:
one or more silica containers;
one or more sensors configured to generate data indicative of one or more characteristics of water at a target location within an aquatic environment; and
a controller configured to:
determine one or more desired parameters for a silica solution based on the one or more characteristics of the water at the target location;
apply a fluid to a silica source within a silica container of the one or more silica containers to wash the silica source, dilute the silica source, or any combination thereof to form the silica solution having the one or more desired parameters; and
generate instructions to distribute the silica solution having the one or more desired parameters into the water at or near the target location.

2. The environmental treatment distribution system of claim 1, wherein the silica source comprises a silica-rich rock, an industrial byproduct, or any combination thereof.

3. The environmental treatment distribution system of claim 1, wherein the silica source comprises an industrial byproduct, and the industrial byproduct comprises a filtered portion of a frac sand.

4. The environmental treatment distribution system of claim 1, wherein the silica source comprises sodium metasilicate pentahydrate, sodium silicate, or any combination thereof.

5. The environmental treatment distribution system of claim 1, wherein the silica source comprises a silica-rich rock, and the controller is configured to apply the fluid to the silica-rich rock to wash the silica-rich rock to release the silica from the silica-rich rock.

6. The environmental treatment distribution system of claim 5, wherein the one or more containers comprise a plurality of silica containers, and the controller is configured to:
receive an indication of a respective source location of the silica-rich rock; and
select the silica-rich rock as the silica source from among a plurality of available silica sources in the plurality of silica containers based on the respective source location of the silica-rich rock being within a threshold vicinity of the target location.

7. The environmental treatment distribution system of claim 1, wherein the one or more characteristics comprise density and pH, and the controller is configured to apply the fluid to the silica source to form the silica solution with a respective density that is within a threshold density range of the density and a respective pH that is within a threshold pH range of the pH.

8. The environmental treatment distribution system of claim 1, wherein the one or more silica containers are supported by an aquatic vessel.

9. The environmental treatment distribution system of claim 8, comprising an output conduit fluidly coupled to at least the silica container of the one or more silica containers, wherein an end portion of the output conduit is positioned to distribute the silica solution to a photic zone of the water.

10. The environmental treatment distribution system of claim 1, wherein the target location comprises an intersection between a tributary and an ocean.

11. The environmental treatment distribution system of claim 1, comprising:
one or more nutrient containers; and
one or more bioreactors;
wherein the controller is configured to generate instructions to distribute the silica solution from the silica container of the one or more silica containers, one or more nutrients from the one or more nutrient containers, and one or more organisms from the one or more bioreactors according to a dispersal program, and the dispersal program comprises respective rates and respective sequential times for distribution of the silica solution, the one or more nutrients, and the one or more organisms to provide a substantially continuous flow of the silica solution, the one or more nutrients, and the one or more organisms into the water at or near the target location.

12. A method of operating an environmental treatment distribution system, the method comprising:
receiving, at one or more processors, one or more characteristics of water at a target location within an aquatic environment;
receiving, at the one or more processors, one or more respective parameters of a plurality of silica sources in a plurality of silica containers supported by an aquatic vessel;
determining, using the one or more processors, one or more desired parameters for a silica solution based on the one or more characteristics of the water at the target location;
selecting, using the one or more processors and based on the one or more characteristics of the water at the target location and the one or more respective parameters of the plurality of silica sources in the plurality of silica containers, a first silica source of the plurality of silica sources;
instructing, using the one or more processors, a processing system to process the first silica source of the plurality of silica sources to adjust the one or more respective parameters of the first silica source of the plurality of silica sources to form the silica solution having the one or more desired parameters; and
instructing, using the one or more processors, distribution of the silica solution into the water at or near the target location.

13. The method of claim 12, wherein the first silica source comprises a silica-rich rock, and the instructing the processing system comprises instructing the processing system to wash the silica-rich rock to form the silica solution.

14. The method of claim 12, wherein the first silica source comprises a solid powder or a liquid solution, and the instructing the processing system comprises instructing the processing system to mix a sample of the water extracted at or near the target location with the first silica source to form the silica solution.

15. The method of claim 14, wherein the first silica source comprises sodium metasilicate pentahydrate as the solid powder or sodium silicate as the liquid solution.

16. The method of claim 12, wherein the determining comprises inputting sensor data indicative of the one or more characteristics of the water at the target location into one or more machine learning algorithms to predict the one or more desired parameters for the silica solution that are expected to provide a desirable effect at or near the target location.

17. The method of claim 12, wherein the instructing the processing system comprises instructing the processing system to process the first silica source of the plurality of silica sources as the aquatic vessel transports the plurality of silica sources in the plurality of silica containers and the processing system toward the target location.

18. The method of claim 12, comprising instructing, using the one or more processors, distribution of the silica solution with iron and one or more organisms, wherein the iron originates from iron cuttings, and the distribution of the silica solution with the iron provides more silica than the iron into the water at or near the target location to mimic naturally occurring volcanic ash or dust.

19. The method of claim 12, wherein the instructing the distribution comprises instructing the distribution of the silica solution over a first period of time, and the method comprises:
receiving, at the one or more processors, one or more updated characteristics of the water at the target location;
determining, using the one or more processors, one or more updated desired parameters for an additional silica solution based on the one or more updated characteristics of the water at the target location;
selecting, using the one or more processors and based on the one or more updated characteristics of the water at the target location and the one or more respective parameters of the plurality of silica sources in the plurality of silica containers, a second silica source of the plurality of silica sources;
instructing, using the one or more processors, the processing system to process the second silica source of the plurality of silica sources to adjust the one or more respective parameters of the second silica source of the plurality of silica sources to form the additional silica solution with the one or more updated desired parameters; and
instructing, using the one or more processors, distribution of the additional silica solution at or near the target location over a second period of time.

20. An environmental treatment distribution system, comprising:
a silica container supported on an aquatic vessel within an aquatic environment;
a controller configured to generate instructions to:
as the aquatic vessel travels within the aquatic environment:
receive one or more characteristics of a target location within the aquatic environment;
determine an arrival time of the aquatic vessel to the target location;
predict one or more desired parameters for a silica solution based on the one or more characteristics of the target location and the arrival time;
control a processing system to apply a fluid to a silica source in the silica container to form the silica solution with the one or more desired parameters; and
in response to the aquatic vessel reaching the target location at or near the arrival time, control one or more output devices to distribute the silica solution into the water at the target location.
